(19) United States
(12) Patent Application Publication
(10) Pub. No.: US 2016/0169766 A1
(43) Pub. Date: Jun. 16, 2016

(54) LEAKAGE DETERMINING METHOD, SUBSTRATE PROCESSING APPARATUS AND STORAGE MEDIUM

(71) Applicant: TOKYO ELECTRON LIMITED, Tokyo (JP)

(72) Inventors: Seiji ISHIBASHI, Yamanashi (JP); Hiromitsu SAKAUE, Yamanashi (JP); Yoshiaki SASAKI, Yamanashi (JP)

(21) Appl. No.: 14/965,684

(22) Filed: Dec. 10, 2015

(30) Foreign Application Priority Data
Dec. 11, 2014 (JP) 2014-251085

(51) Int. Cl. G01M 3/02; H01L 21/67; G01N 33/00

(52) U.S. Cl. CPC G01M 3/02; G01N 33/0036; H01L 21/67196; H01L 21/67253; H01L 21/67098

(57) ABSTRACT

A leakage determining method determines whether or not atmospheric air enters a vacuum transfer chamber for transferring a substrate under a vacuum atmosphere between a preliminary vacuum chamber and a processing chamber. The method includes controlling a pressure in the vacuum transfer chamber to a preset pressure by supplying a pressure control gas into the vacuum transfer chamber; performing supply control, when the substrate is not transferred, by reducing the amount of the pressure control gas supplied into the vacuum transfer chamber or stopping the supply of the pressure control gas; and measuring an oxygen concentration in the vacuum transfer chamber after the supply control of the pressure control gas and determining leakage of atmospheric air into the vacuum transfer chamber by determining whether or not atmospheric air whose amount exceeds a preset allowable level enters the vacuum transfer chamber based on temporal changes of the measured oxygen concentration.

### CROSS-REFERENCE TO RELATED APPLICATION

This application claims priority to Japanese Patent Application No. 2014-251085 filed on Dec. 11, 2014, the entire contents of which are incorporated herein by reference.

### FIELD OF THE INVENTION

The disclosure relates to a technique for determining whether or not atmospheric air enters a vacuum transfer chamber where a substrate is transferred under a vacuum atmosphere.

### BACKGROUND OF THE INVENTION

In a semiconductor device manufacturing process, there are used various processing modules for processing a wafer in a processing chamber set to a vacuum atmosphere, such as a film forming module for forming a film by reaction of a reactant gas on a surface of a semiconductor wafer (hereinafter, referred to as “wafer”), a plasma processing module for processing the film formed on the surface of the wafer by using a plasma, and the like. Further, there is known a substrate processing apparatus referred to as a multi-chamber type apparatus or a cluster tool type apparatus in which a plurality of processing modules is connected to a vacuum transfer chamber where a wafer is transferred under a vacuum atmosphere.

Such a substrate processing apparatus includes a load-lock chamber where a wafer to be transferred between an outside and a vacuum transfer chamber is accommodated and is loaded or unloaded after an inner atmosphere of the load-lock chamber is switched between an atmospheric atmosphere and a vacuum atmosphere.

The vacuum transfer chamber is connected to the processing modules and the load-lock chambers via gate valves. In order to prevent a pressure from being changed when the gate valves are opened/closed, a pressure in the vacuum transfer chamber is controlled.

As a technique for controlling a pressure in the vacuum transfer chamber, there is known a technique for supplying an inert gas for pressure control into the vacuum transfer chamber while exhausting the vacuum transfer chamber by a vacuum pump or the like and increasing/decreasing a gas supply amount such that a pressure in the vacuum transfer chamber becomes close to a set level.

However, when atmospheric air in the outside enters (leaks into) the vacuum transfer chamber through connection portions with the processing modules or the load-lock modules, an oxygen concentration (oxygen partial pressure) may be increased in a state where a pressure condition (total pressure) in the vacuum transfer chamber is maintained at a proper state. Conventionally, components contained in a vacuum atmosphere are not managed in the vacuum transfer chamber where only the transfer of the wafer is performed.

Japanese Patent Application Publication No. 2006-261296 (claim 1, paragraphs [0028] to [0030], FIG. 3) discloses a technique for performing a purge process by supplying nitrogen gas into a processing chamber where a wafer is subjected to heat treatment using hydrogen gas and performing the heat treatment by introducing the hydrogen gas after an oxygen concentration in the processing chamber becomes lower than a tolerance value. In addition, Japanese Patent Application Publication No. 2013-201292 (paragraphs 0050 and 0081 to 0089, FIG. 2) discloses a technique for evacuating a processing chamber while supplying an inert gas into the processing chamber until a pressure in the processing chamber reaches a level substantially the same as the atmospheric pressure, measuring an oxygen concentration in the processing chamber while sealing the processing chamber, determining that there is no leakage in the processing chamber when the measurement result is smaller than a predetermined maximum level, and performing heat treatment of a wafer.

However, there is no description in any of Japanese Patent Application Publication Nos. 2006-261296 and 2013-201292 that a vacuum transfer chamber is provided outside the processing chamber. Further, there is no description on a technique for determining whether or not leakage occurs in the vacuum transfer chamber of which inner pressure is controlled by a pressure control gas.

### SUMMARY OF THE INVENTION

In view of the above, the disclosure provides a leakage determining method for determining whether or not atmospheric air enters a vacuum transfer chamber to which a pressure control gas is supplied, a substrate processing apparatus, and a storage medium in which the method is stored.

In accordance with an aspect of present invention, there is provided a leakage determining method for determining whether or not atmospheric air enters a vacuum transfer chamber for transferring a substrate under a vacuum atmosphere between at least one preliminary vacuum chamber and at least one processing chamber, the vacuum transfer chamber being connected to the preliminary vacuum chamber of which inner atmosphere is switchable between an atmospheric atmosphere and a vacuum atmosphere and to the processing chamber where the substrate is processed under a vacuum atmosphere, via respective opening/closing valves, the method including: controlling, when the substrate is transferred, a pressure in the vacuum transfer chamber to a preset pressure by supplying a pressure control gas into the vacuum transfer chamber being evacuated; performing supply control, when the substrate is not transferred, by reducing the amount of the pressure control gas supplied into the vacuum transfer chamber or stopping the supply of the pressure control gas; and measuring with an oxygen meter an oxygen concentration in the vacuum transfer chamber after the supply control of the pressure control gas and determining leakage of atmospheric air into the vacuum transfer chamber by determining whether or not atmospheric air whose amount exceeds a preset allowable level enters the vacuum transfer chamber based on temporal changes of the measured oxygen concentration.

### BRIEF DESCRIPTION OF THE DRAWINGS

The objects and features of the disclosure will become apparent from the following description of embodiments, given in conjunction with the accompanying drawings, in which:

FIG. 1 is a top view of a substrate processing apparatus according to an embodiment;

FIG. 2 is a vertical cross sectional side view of a vacuum transfer chamber of the substrate processing apparatus;

FIG. 3 is a flowchart of an operation of determining leakage into the vacuum transfer chamber;

FIG. 4 is a horizontal top view of the vacuum transfer chamber in a state where a wafer transfer process is performed;

FIG. 5 is a horizontal top view of the vacuum transfer chamber in a state where a leakage determining process is performed;

FIG. 6 is a horizontal top view of the vacuum transfer chamber in a state where a process of determining leakage into processing modules is performed;

FIG. 7 is a horizontal top view of the vacuum transfer chamber in a state where a process of determining leakage into load-lock chambers is performed;

FIG. 8 is a flowchart of an operation of determining leakage into a vacuum transfer chamber in accordance with another example;

FIG. 9 explains a pressure in the vacuum transfer chamber and temporal changes in an oxygen concentration in the case of varying a leakage amount;

FIG. 10 explains relation between a leakage amount and an oxygen concentration in the case of varying a set pressure in the vacuum transfer chamber; and

FIG. 11 explains relation between a set pressure and an oxygen concentration in the case of varying a leakage amount of atmospheric air into the vacuum transfer chamber.

### DETAILED DESCRIPTION OF THE EMBODIMENTS

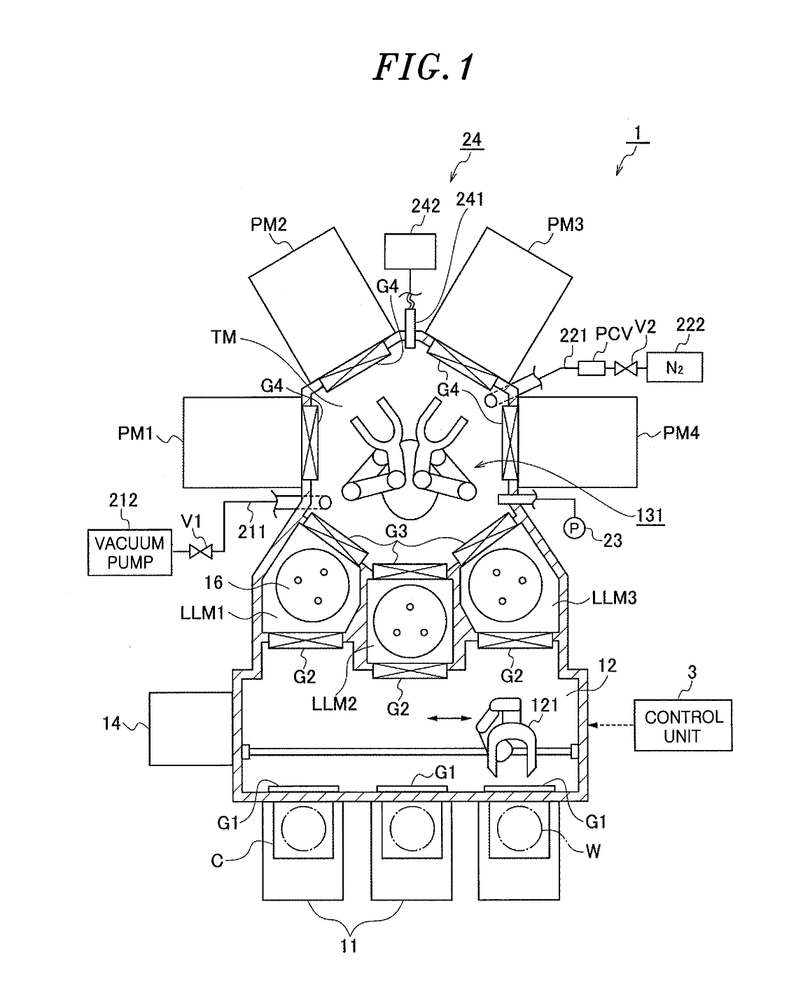

The following is description on an example of a substrate processing apparatus 1 according to an embodiment which includes a plurality of processing modules PM1 to PM4 for forming a film on a wafer as a substrate by using a CVD (Chemical Vapor Deposition) method or an ALD (Atomic Layer Deposition) method. As shown in FIG. 1, the substrate processing apparatus 1 includes: a carrier mounting stage 11 for mounting thereon a carrier C accommodating a predetermined number (e.g., 25 sheets) of wafers W to be processed; an atmospheric transfer chamber 12 for transferring a wafer W unloaded from the carrier C under an atmospheric atmosphere; load-lock chambers (preliminary vacuum chambers) LLM1 to LLM3 where the wafer W waits and an atmosphere therein is switched between an atmospheric atmosphere and a preliminary vacuum atmosphere (vacuum atmosphere); a vacuum transfer chamber TM for transferring the wafer W under a vacuum atmosphere; and processing modules PM1 to PM4 for processing the wafer W. These components are arranged, whenseen from the loading direction of the wafer W, in the order of the atmospheric transfer chamber 12, the load-lock chambers LLM1 to LLM3, the vacuum transfer chamber TM, and the processing modules PM1 to PM4. The components adjacent to each other are airtightly connected via a door G1, a door valve G2 or gate valves G3 and G4. The gate valves G3 and G4 respectively correspond to an opening/closing valve disposed between the load-lock chambers LLM1 to LLM3 and the vacuum transfer chamber TM and opening/closing valves disposed between the vacuum transfer chamber TM and the processing modules PM1 to PM4.

Provided in the atmospheric transfer chamber 12 is a rotatable, extensible/contractible, and vertically/horizontally movable transfer arm 121 for unloading wafers W one at a time from the carrier C. Provided at a side of the atmospheric transfer chamber 12 is an alignment chamber 14 having an orienter for positioning the wafer W.

Three load-lock chambers LLM1 to LLM3 are arranged in a left and right direction when seen from the carrier mounting stage 11 side. The atmospheric transfer chamber 12 and the vacuum transfer chamber TM are connected via the load-lock chambers LLM1 to LLM3. Provided in each of the load-lock chambers LLM1 to LLM3 is a mounting table 16 having supporting pins for supporting the loaded wafer W from the bottom surface side thereof. A vacuum pump (not shown) or a leakage valve (not shown) for switching an inner atmosphere of each load-lock chamber between an atmospheric atmosphere and a preliminary vacuum atmosphere is connected to each of the load-lock chambers ULM1 to LLM3.

Each of the load-lock chambers LLM1 to LLM3 is used for loading/unloading wafers W. In the case of unloading a wafer W, a wafer W mounted on the supporting pins is cooled while waiting for a predetermined period of time in any one of the load-lock chambers LLM1 to LLM3 of which inner atmosphere has been switched to an atmospheric atmosphere.

The vacuum transfer chamber TM has a heptangular shape when seen from the top. An inner space of the vacuum transfer chamber TM is set to a vacuum atmosphere. The load-lock chambers LLM1 to LLM3 are connected to front three sides of the vacuum transfer chamber TM. The processing modules PM1 to PM4 are connected to the other four sides of the vacuum transfer chamber TM. Provided in the vacuum transfer chamber TM is a rotatable and extensible/contractible transfer arm 131 for transferring the wafer W between the load-lock chambers LLM1 to LLM3 and the processing modules PM1 to PM4.

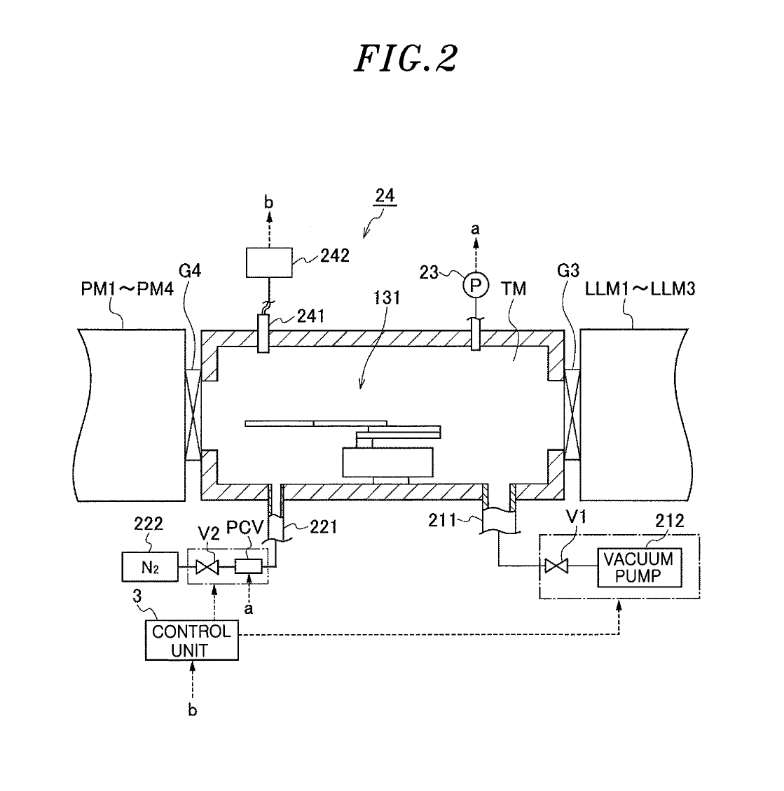

As illustrated in FIGS. 1 and 2, the vacuum transfer chamber TM is connected to a gas exhaust line 211 for evacuating the vacuum transfer chamber TM. A vacuum pump 212 is disposed at a downstream side of the gas exhaust line 211 via an opening/closing valve V1. The vacuum transfer chamber TM is connected to a nitrogen gas supply line 221 for supplying an inert gas, e.g., nitrogen gas, serving as a pressure control gas into the vacuum transfer chamber TM. A pressure control valve PCV is disposed in the nitrogen gas supply line 221. A nitrogen gas supply unit 222 is provided at an upstream side of the nitrogen gas supply line 221 via an opening/closing valve V2.

The pressure control valve PCV has a function of controlling a pressure by increasing/decreasing a nitrogen gas supply amount such that a pressure in the vacuum transfer chamber TM becomes close to a preset value based on a difference value between the preset value and a measured value of a pressure gauge 23 provided at the vacuum transfer chamber TM.

In the processing modules PM1 to PM4 of the substrate processing apparatus 1 of the present embodiment, a common film forming process is performed on the wafer W. The wafer W transferred in the vacuum transfer chamber TM is loaded into one of the processing modules PM1 to PM4 which is in a waiting mode without forming a film on another wafer W, and then subjected to a film forming process in the corresponding processing module. Each of the processing modules PM1 to PM4 is configured as a film forming module for forming a film on the wafer W by supplying a processing gas, the wafer W being mounted on a mounting table (not shown) provided in a processing chamber (processing container) of a vacuum atmosphere and heated thereon.

The wafer W in the processing modules PM1 to PM4 is heated to, e.g., about several hundreds of ° C. The film forming process is performed by reaction of the processing gas supplied to the surface of the wafer W. The type of the film forming process performed in the processing modules PM1 to PM4 is not particularly limited. It may be a CVD method in which film formation reaction proceeds by supplying a source gas onto the surface of the heated wafer W or may be an ALD method in which a laminated film is formed by repeating a process of allowing a source gas to be adsorbed onto the surface of the wafer W and a process of forming an atomic layer or a molecular layer of a reaction by-product by supplying a reactant gas that reacts with the source gas. The wafer W may be heated by a heater provided at the mounting table on which the wafer W is mounted. Or, there may be employed a hot-wall type method using a heater provided at a wall of the processing chamber. A plasma generation unit for turning a processing gas into a plasma may be provided at the processing modules PM1 to PM4 and an activated processing gas may be supplied to the wafer W.

As shown in FIGS. 1 and 2, the substrate processing apparatus I includes a control unit 3. The control unit 3 is configured as a computer having a CPU (Central Processing Unit) and a storage unit (both not shown). The storage unit stores therein a program having a group of steps (commands) for outputting a control signal for executing the above-described processes for processing the wafer W. The program is stored in a storage medium, e.g., a hard disk, a compact disk, a magneto-optical disc, a memory card or the like, and installed in the storage unit.

The substrate processing apparatus 1 configured as described above includes an oxygen meter 24 for measuring an oxygen concentration of an inner atmosphere of the vacuum transfer chamber TM and determines whether or not the amount of atmospheric air that enters the vacuum transfer chamber TM from the outside (hereinafter, may be referred to as “leakage”) exceeds a preset tolerable amount based on the oxygen concentration measured by the oxygen meter 24.

Hereinafter, the reason for determining the leakage into the vacuum transfer chamber TM will be described. As described above, a pressure in the vacuum transfer chamber TM is controlled and maintained at a substantially constant level (close to a preset level) by using nitrogen gas. Conventionally, whether or not atmospheric air whose amount exceeds an allowable level leaks into the vacuum transfer chamber TM is determined based on the pressure (total pressure) in the vacuum transfer chamber TM.

Specifically, when the wafer W is not processed in the processing modules PM1 to PM4, the supply of the nitrogen gas for pressure control is stopped (the opening/closing valve V2 is closed) and, then, the vacuum transfer chamber TM is evacuated by the vacuum pump 212. When the saturation state in which the pressure in the vacuum transfer chamber TM is no longer decreased is reached, the evacuation is stopped and the opening/closing valve V1 of the vacuum pump 212 side is closed. In that state, temporal changes of the measured value of the pressure gauge 23 are monitored. When the measured value of the pressure gauge 23 reaches a maximum level of the preset pressure within a predetermined period of time, it is determined that the leakage of which amount exceeds an allowable level occurs.

Such a technique can detect the leakage of about 0.9 seem in the vacuum transfer chamber TM having a volume of, e.g., about 150 liters. However, it is difficult to detect a leakage of a smaller amount. If the leakage is determined frequently and ten to several tens of minutes are taken to perform a single leakage determining process, the operation rate of the substrate processing apparatus 1 may deteriorate.

With respect to the wafer W transferred in the vacuum transfer chamber TM, as the film formed on the wafer W becomes thinner, it is required to accurately determine the leakage into the vacuum transfer chamber TM.

Hereinafter, an effect of the leakage in the case of forming a metal film on the wafer W in the processing modules PM1 to PM4 will be described as an example. Conventionally, heat radiation due to contact with the transfer arm 131 or an ambient atmosphere hardly occurs in the wafer W transferred under a high vacuum atmosphere. Therefore, the wafer W is transferred to the load-lock chambers LLM1 to LLM3 at a temperature substantially the same as that of the wafer W unloaded from the processing modules PM1 to PM4.

When a pressure in the vacuum transfer chamber TM is set to a range from about 10 Pa to about 1333 Pa, nitrogen gas for pressure control serves as a heat transfer gas and the wafer W radiates heat to the transfer arm 131. As a result, the temperature in the surface of the wafer W transferred in the vacuum transfer chamber TM is lower at a contact portion with the transfer arm 131 (including a portion close to the transfer arm 131 without contact therewith) than at the other portions. In a region where the pressure is less than 10 Pa, the mean free path of the gas in the vacuum transfer chamber TM is long, so that the heat transfer by the gas hardly occurs.

The present inventors have found that the metal film is easily oxidized when the temperature of the wafer W ranges from about 200° C. to about 300° C. compared to when it is about 400° C. or above. In the load-lock chambers LLM1 to LLM3 where the wafer W is cooled under an atmospheric atmosphere, the wafer W passes through the above temperature range within a few seconds. However, in the vacuum transfer chamber TM, the wafer W is not actively cooled and, thus, a longer period of time is taken for the wafer W to pass through the above temperature range.

The wafer W that has been subjected to the film formation and transferred in the vacuum transfer chamber TM may have a temperature at which oxidation easily occurs for a relatively long period of time. If atmospheric air in the outside enters the vacuum transfer chamber TM where the wafer W having such a temperature is transferred, the metal film is oxidized at the contact portion with the transfer arm 131 having a low temperature (including a portion close to the transfer arm 131 without contact therewith). As a result, the in-plane resistivity uniformity of the metal film may deteriorate or the total resistivity of the metal film may increase.

Atmospheric air is prone to leak into the vacuum transfer chamber TM through sealing surfaces between the vacuum transfer chamber TM and the gate valves G4 where a temperature is increased due to heat transferred from the processing modules PM1 to PM4, bellows portions of the gate valves G3 and G4 where sliding or abrasion of a driving unit occurs, or the like. The atmospheric air may also leak into the processing modules PM1 to PM4 and the load-lock chambers LLM1 to LLM3, and oxygen may enter the vacuum transfer chamber TM when the gate valves G3 and G4 are opened.

From the above, it is clear that even a small amount of leakage which does not affect the control of a pressure in the vacuum transfer chamber TM needs to be checked and also that the leakage into the vacuum transfer chamber TM needs to be determined within a short period of time which does not affect the operation of the substrate processing apparatus 1.

As shown in FIGS. 1 and 2, the oxygen meter 24 is provided at the vacuum transfer chamber TM to determine leakage based on the measured oxygen concertation in the vacuum transfer chamber TM in this example. The type of the oxygen meter 24 is not particularly limited. However, in this example, there is employed a zirconia oxygen meter 24 for measuring oxygen gas concentration in a measurement gas based on an electromotive power generated when zirconia is made to contact with oxygen gases having different concentrations (measurement gas and comparison gas).

Although a single oxygen meter 24 is illustrated in the drawing, there may be provided a plurality of oxygen meters 24. In a viscous flow region where the pressure is, e.g., about 10 Pa or above, a pressure in the vacuum transfer chamber TM is non-uniform even under a vacuum atmosphere. Therefore, a region where a pressure is relatively high and a region where a pressure is relatively low exist and the pressure distribution is non-uniform. Since the pressure distribution may affect the oxygen concentration distribution, a plurality of pressure gauges 23 and a plurality of oxygen meters 24 are provided at the vacuum transfer chamber TM to quickly and accurately perform leakage determination even when non-uniform oxygen concentration distribution exists.

The oxygen meter 24 includes a sensor unit 241 having an electrode provided at zirconia ceramic, and a main body 242 for detecting an electromotive force taken out from the electrode as a potential difference by a voltmeter and converting the detected potential difference to an oxygen concentration. The oxygen concentration in the vacuum transfer chamber TM which is measured by the oxygen meter 24 is outputted to the control unit 3 (see FIG. 2).

The oxygen meter 24 can perform leakage determination by measuring the oxygen concentration in a state where the gate valves G3 and G4 disposed between the load-lock chambers LLM1 to LLM3 and the vacuum transfer chamber TM and between the processing modules PM1 to PM4 and the vacuum transfer chamber TM are opened.

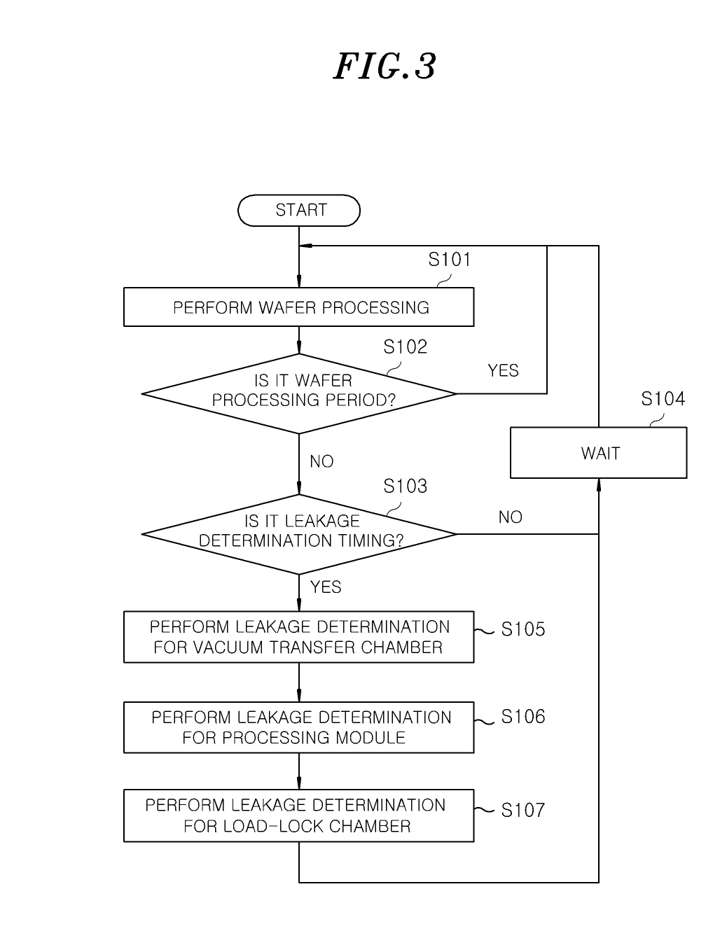

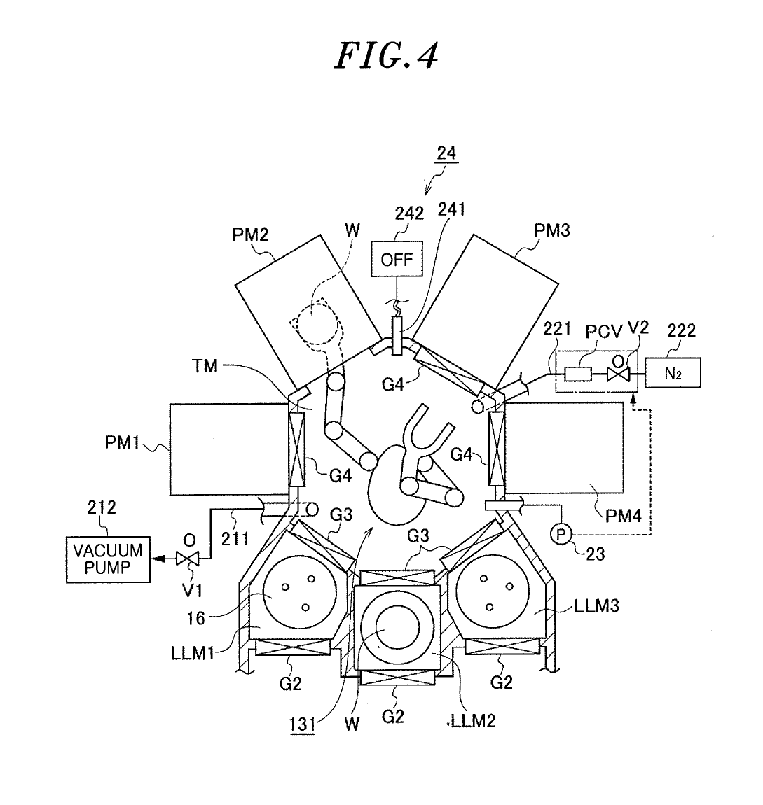

Hereinafter, an operation of the substrate processing apparatus 1 of the present embodiment will be described with reference to the flowchart of FIG. 3 and the process diagrams of FIGS. 4 to 7.

When the substrate processing apparatus 1 starts to operate (start in FIG. 3), the wafer W is normally subjected to a film forming process (step S101 in FIG. 3). In other words, when the carrier C accommodating wafers W is mounted on the carrier mounting stage 11, the wafers W in the carrier C are unloaded one by one by the transfer arm 121. The wafer W held on the transfer arm 121 is positioned in the alignment chamber 14 during the transfer in the atmospheric transfer chamber 12 and, then, transferred to one of the load-lock chambers LLM1 to LLM3 for loading (e.g., LLM1).

When the load-lock chamber LLM1 is under a preliminary vacuum atmosphere, the wafer W is taken by the transfer arm 131 and loaded into the vacuum transfer chamber TM. Thereafter, the wafer W is loaded into any one of the processing modules PM1 to PM4 which can accommodate the wafer W and, then, subjected to a predetermined film forming process (see FIG. 4). The wafer W that has been subjected to the film forming process is transferred into one of the load-lock chambers LLM1 to LLM3 via the vacuum transfer chamber TM and cooled under an atmospheric atmosphere. Next, the wafer W is transferred to the atmospheric transfer chamber 12 and accommodated in the original carrier C.

During the above processing period, the vacuum transfer chamber TM is evacuated by the vacuum pump 212 and the pressure is controlled by increasing/decreasing the supply amount of the nitrogen gas based on the pressure in the vacuum transfer chamber TM which is detected by the pressure gauge 23, as can be seen from FIG. 4. Further, during the above processing period, the leakage determination using the oxygen meter 24 is not performed (“OFF” state of the main body 242 in FIG. 4).

During the period in which the wafer W is subjected to the film forming process (step S102 in FIG. 3; YES), the processing of the wafer W is continued (step S101). During the period in which the wafer W is not processed (step S102; NO), it is determined whether or not the leakage determination is required (step S103).

If a preset timing of leakage determination has not come even during the period in which the film forming process is not performed, (step S103; NO), the wafer W waits for restart of the processing (step S104).

If the preset timing of leakage determination has elapsed (step S103; YES), the leakage determination for the vacuum transfer chamber TM is performed (step S105).

The timing of leakage determination is preset by the control unit 3 of the substrate processing apparatus 1. Specifically, it is set such that next leakage determination is performed after a predetermined period of time elapses from previous leakage determination (e.g., after a day or a week elapses from the previous leakage determination) or after a predetermined number of wafers N are processed.

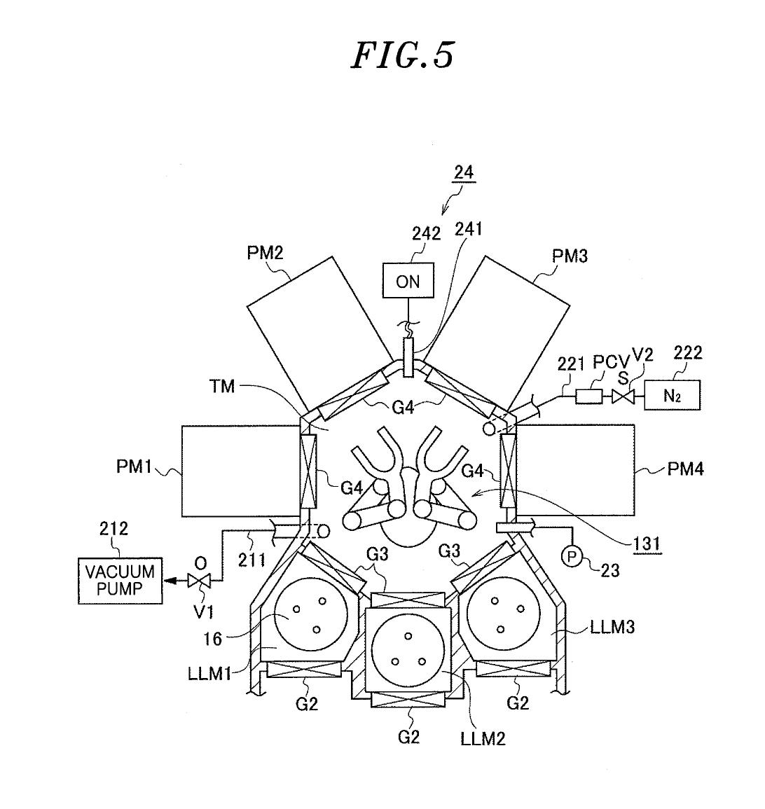

In order to perform the leakage determination for the vacuum transfer chamber TM, the gate valves G3 and G4 disposed between the load-lock chambers LLM1 to LLM3 and the vacuum transfer chamber TM and between the processing modules PM1 to PM4 and the vacuum transfer chamber TM are closed and the vacuum transfer chamber TM is isolated from the load-lock chambers LLM1 to LLM3 and the processing modules PM1 to PM4, as can be seen from FIG. 5. The nitrogen gas supply from the nitrogen gas supply unit 222 is stopped while continuing the evacuation operation of the vacuum pump 212, and the oxygen concentration in the vacuum transfer chamber TM is measured by the oxygen meter 24 (“ON” state of the main body 242 in FIG. 5).

As will be described in the following test results of test examples, when the supply of the nitrogen gas is stopped, the dilution effect of the nitrogen gas disappears. Therefore, if atmospheric air leaks into the vacuum transfer chamber TM, the oxygen concentration measured by the oxygen meter 24 is increased. When the oxygen concentration reaches a preset maximum level within a predetermined period of time, it is determined that atmospheric air whose amount exceeds an allowable level leaks into the vacuum transfer chamber TM. According to the following test results, the leakage determination can be performed within, e.g., a few minutes.

If the supply of the nitrogen gas is stopped during the transfer of the wafer W, the oxidation of the film may be facilitated as the oxygen concentration in the vacuum transfer chamber TM is increased. Therefore, it is not preferable to perform a process of measuring an oxygen concentration in the vacuum transfer chamber TM which requires stopping of the supply of the nitrogen gas during the transfer of the wafer W.

After the leakage determination for the vacuum transfer chamber TM is performed, the leakage determination for the processing modules PM1 to PM4 is performed (step S106 in FIG. 3).

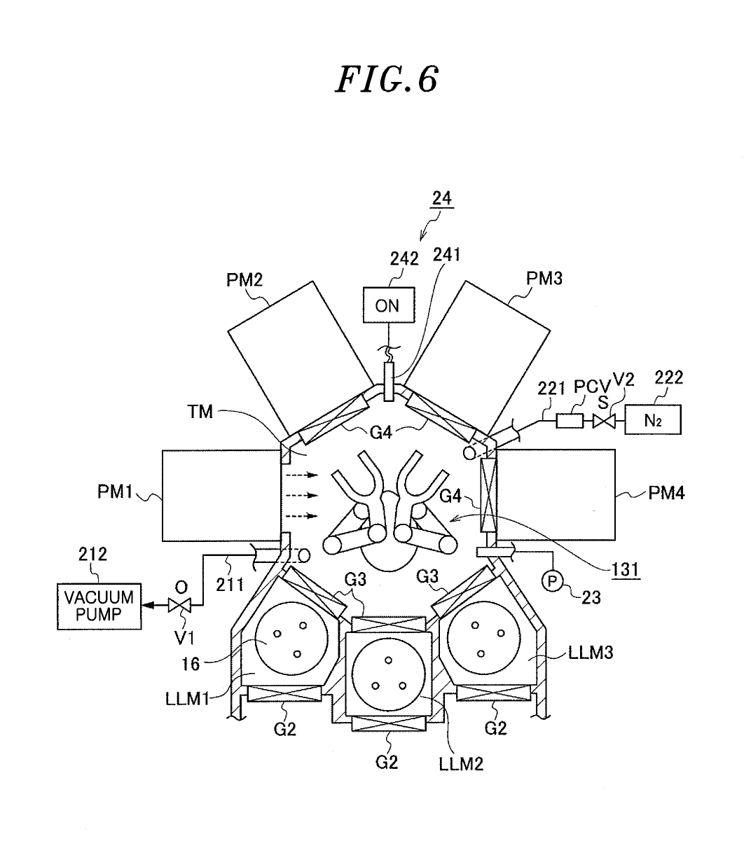

In order to perform the leakage determination for the processing modules PM1 to PM4, the evacuation operation of the vacuum pump 212 and the operation of stopping the nitrogen gas supply are carried out as in the case of performing the leakage determination for the vacuum transfer chamber TM. Then, for example, the processing module PM1 and the vacuum transfer chamber TM are made to communicate with each other by opening the gate valve G4 of the processing module PM1 (see FIG. 6).

At this time, if there is leakage into the processing module PM1, atmospheric air that has entered the processing module PM1 is introduced into the vacuum transfer chamber TM and an increase in the oxygen concentration is monitored. When the oxygen concentration reaches a preset maximum level within a predetermined period of time, it is determined that the atmospheric air whose amount exceeds an allowable level leaks into the vacuum transfer chamber TM via the processing module PM1.

After the leakage determination for the processing module PM1 is completed, the leakage determination for each of the other processing modules PM2 to PM4 is performed in the same manner as that for the processing module PM1, by sequentially opening the gate valves G4 of the other processing modules PM2 to PM4 one by one.

The sequence of the process of performing leakage determination for the processing modules PM1 to PM4 is not limited to that described in the above example. For example, the leakage determination is performed by opening all of the gate valves G4 of the processing modules PM1 to PM4 at once. If the increase in the oxygen concentration is monitored and thus it is determined that there is leakage, a processing module where the leakage occurs may be specified among the processing modules PM1 to PM4 by opening the gate valves G4 of the processing module PM1 to PM4 one by one. When there is no leakage, the subsequent leakage determination process is not required and, thus, an average time of the leakage determination can be shortened.

After the leakage determination for the processing modules PM1 to PM4 is performed, the leakage determination for the load-lock chambers LLM1 to LLM3 is performed (step 5107 in FIG. 3).

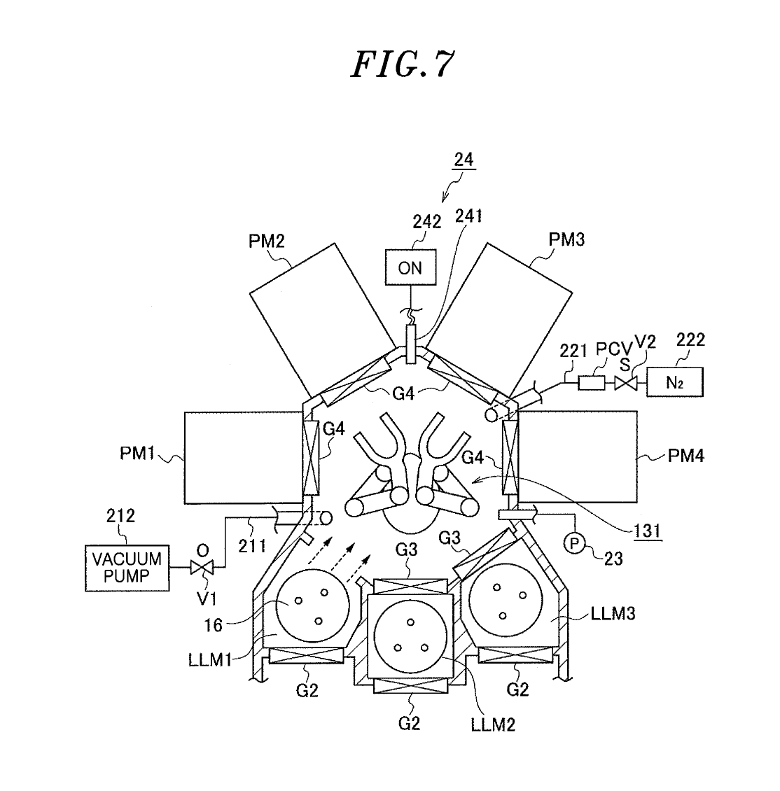

The leakage determination for the load-lock chambers LLM1 to LLM3 is performed by opening the gate valves G3 of the load-lock chambers LLM1 to LLM3 one by one in the same manner as that used in performing the leakage determination for the processing modules PM1 to PM4 (see FIG. 7). At this time, the leakage determination for the load-lock chambers LLM1 to LLM3 is performed in a state where an inner atmosphere thereof is set to a preliminary vacuum atmosphere by closing the door valves G2 of the atmospheric transfer chamber 12 side.

Also in the leakage determination for the load-lock chambers LLM1 to LLM3, if it is determined that there is leakage after performing the leakage determination by opening all the gate valves G3 at once, a load-lock chamber where the leakage occurs may be specified among the load-lock chambers LLM1 to LLM3 by opening the gate valves G3 one at a time.

In this manner, the leakage determination for the vacuum transfer chamber TM, the processing modules PM1 to PM4, and the load-lock chambers LLM1 to LLM3 is completed. When there is leakage, the corresponding device is specified and alarm is generated. As a result, a maintenance staff specifies a portion where the leakage occurs by using a leakage checker and performs a required process such as bolt tightening, packing exchange or the like. When there is no leakage, the wafer W waits for restart of the processing (step S104 in FIG. 3). In the above description, the steps S105 to S107 were executed sequentially. However, any one of the steps S105 to S107 may be executed.

The substrate processing apparatus 1 according to the present embodiment has the following effects. The oxygen concentration in the vacuum transfer chamber TM where the wafer W is transferred under a vacuum atmosphere can be measured while suppressing the dilution effect of the nitrogen gas, because the oxygen concentration in the vacuum transfer chamber TM is measured by the oxygen meter 24 after the supply of the nitrogen gas for pressure control to the vacuum transfer chamber TM is stopped. As a result, it is possible to quickly determine whether or not atmospheric air whose amount exceeds an allowable level enters the vacuum transfer chamber TM.

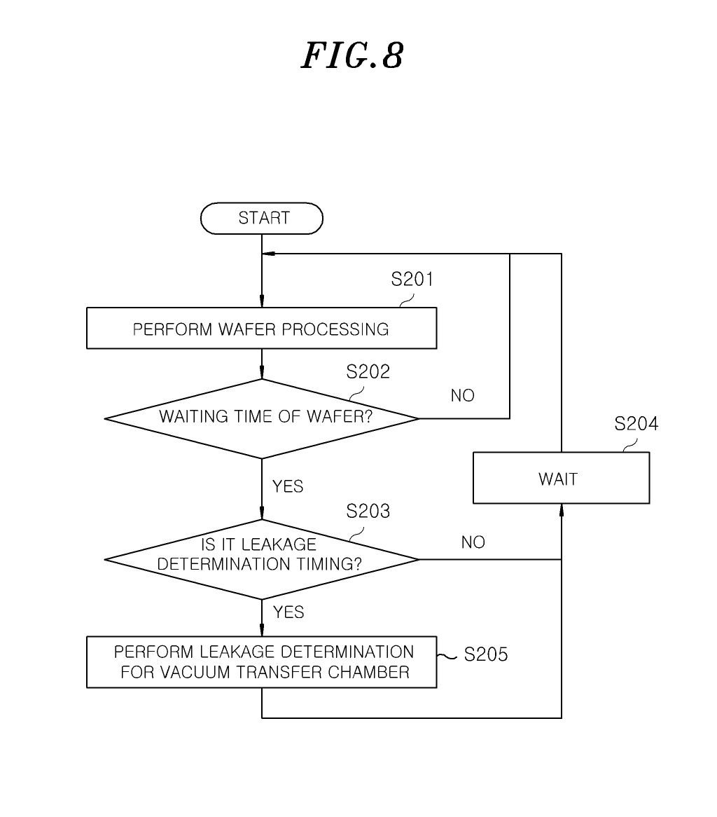

The timing of performing the leakage determination for the vacuum transfer chamber TM is not limited to the timing of stopping the processing of the wafer W as in the case of the example described with reference to FIG. 3. For example, as shown in the flowchart of FIG. 8, the leakage determination for the vacuum transfer chamber TM may be performed (step S205) when the wafer W waits without being transferred in the vacuum transfer chamber TM during the processing period (step S201) and when the waiting time is longer than a period of time required for the leakage determination (step S202; YES) and elapses the timing of the leakage determination (step S203; YES).

A specific technique of the leakage determination of this example is the same as that described with reference to FIG. 5. Since, however, the wafer W that is being processed may be accommodated in the processing modules PM1 to PM4 or the load-lock chambers LLM1 to LLM3, only the leakage determination for the vacuum transfer chamber TM is performed. In the case of performing the leakage determination for the vacuum transfer chamber TM, if there are unused devices among the processing modules PM1 to PM4 and the load-lock chambers LLM1 to LLM3 and the leakage determination can be completed within the waiting time, the leakage determination for the unused devices among the processing modules PM1 to PM4 and the load-lock chambers LLM1 to LLM3 can also be performed by the technique described with reference to FIGS. 6 and 7.

When the leakage determination is performed, it is not necessary to stop the supply of the nitrogen gas for pressure control. For example, the increase in the oxygen concentration due to the leakage is monitored by the oxygen meter 24 when an average of the oxygen concentration in a gas flowing into the vacuum transfer chamber TM, the gas including the nitrogen gas of which amount has been reduced to a predetermined amount and leakage of atmospheric air into the vacuum transfer chamber TM, is higher than the oxygen concentration in the vacuum transfer chamber TM which is measured before the supply amount of the nitrogen gas is reduced.

It is not necessary to continue the evacuation operation of the vacuum pump 212. For example, the leakage determination may be performed in a state where the evacuation operation is stopped when the supply of the nitrogen gas is stopped (the opening/closing valves V1 and V2 of the exhaust line 211 and the nitrogen gas supply line 221 are closed) and the vacuum transfer chamber TM is sealed.

It is not necessary to perform the leakage determination using the oxygen meter 24 after the supply of the nitrogen gas for pressure control is stopped or after the supply amount of the nitrogen gas is decreased. As will be described in the following test examples, the leakage determination can be performed without stopping the supply of the nitrogen gas or reducing the supply amount of the nitrogen gas by obtaining in advance the set pressure in the vacuum transfer chamber TM, the leakage amount of the atmospheric air, and the oxygen concentration under such conditions. In that case, it is not required to reduce the supply amount of the nitrogen gas in order to perform the leakage determination. Therefore, the leakage determination can be performed while transferring the wafer W in the vacuum transfer chamber TM.

In the above embodiment, the process of forming a metal film or the like has been described as an example of a process performed in the processing modules PM1 to PM4. However, the process performed in the processing modules PM1 to PM4 is not limited thereto. For example, there may be provided a processing module for performing a nitriding process for nitriding a thin film on a surface of a wafer W by performing plasma treatment while supplying ammonia gas, an annealing process for heating the wafer W, an etching process for removing the thin film on the surface of the wafer W, or a plasma ashing process for decomposing and removing a resist film on the surface of the wafer W by the plasma. After completion of the above processes, if the characteristics of the thin film formed on the surface of the wafer W is changed by the effect of moisture in the atmospheric air or oxygen introduced into the vacuum transfer chamber TM during the transfer of the wafer W in the vacuum transfer chamber TM, it is possible to quickly recognize whether the thin film deterioration condition is formed or not, by the leakage determination.

The number, the processing type or the combination of the processing modules PM1 to PM4 or the load-lock chambers LLM1 to LLM3 in the substrate processing apparatus 1 may vary, if necessary. For example, different processes may be performed in the processing modules PM1 to PM4, and wafers W may be loaded into the processing modules PM1 to PM4 in a preset order and processed therein.

(Test Examples)

(Test 1)

In a vacuum transfer chamber TM having a volume of about 150 liters, temporal changes of a pressure and an oxygen concentration in the vacuum transfer chamber TM were measured while varying a (simulated) leakage amount of atmospheric air or a condition for controlling start and stop of supply of the nitrogen for pressure control.

A. Test Condition

Nitrogen gas was supplied into the evacuated vacuum transfer chamber TM while setting a pressure to 100 Pa, and leakage of atmospheric air through a line connected to the vacuum transfer chamber TM was varied to five levels, i.e., 5 sccm, 3 sccm, 1 sccm, 0.1 sccm, and 0 sccm. Under the respective conditions, the supply of the nitrogen was stopped after a predetermined period of time. The oxygen concentration was measured by a zirconia oxygen meter 24.

B. Test Result

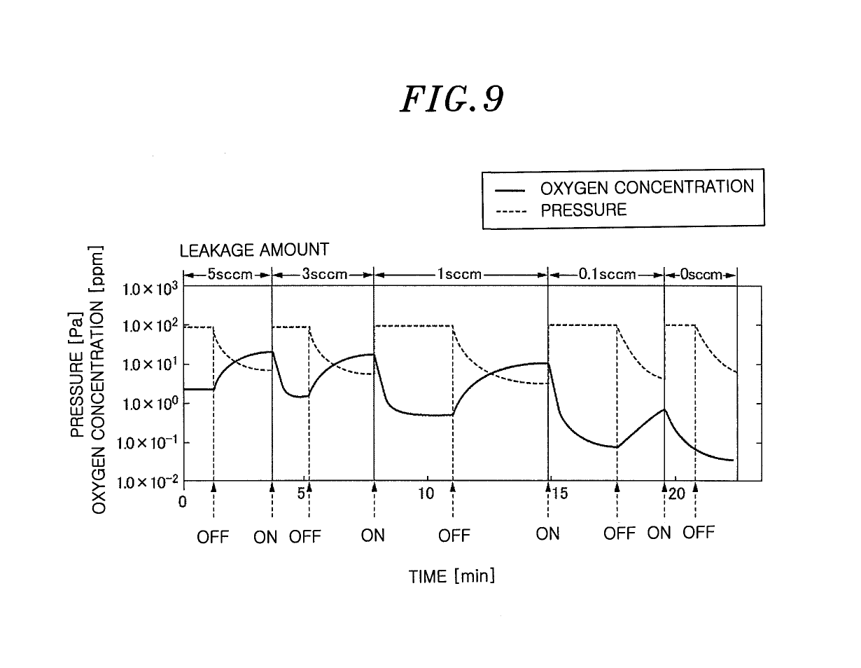

A test result is shown in FIG. 9. In FIG. 9, the horizontal axis represents time (min) and the vertical axis represents a pressure (Pa) or an oxygen concentration (ppm) in the vacuum transfer chamber TM. In FIG. 9, a solid line indicates temporal changes of the oxygen concentration in the vacuum transfer chamber TM and a dashed line indicates temporal changes of the pressure. In the horizontal axis of FIG. 9, the timing of stopping the supply of the nitrogen gas for pressure control is expressed as “OFF” and the timing of restarting the supply of the nitrogen gas is expressed as “ON”.

According to the result shown in FIG. 9, even if the leakage amount is changed, the pressure in the vacuum transfer chamber TM is maintained substantially at the set level as long as the nitrogen gas for pressure control is supplied. It was monitored that, under any condition of the leakage amount (5 sccm, 3 sccm, 1 sccm, and 0.1 sccm), the oxygen concentration was increased immediately after the stop of supplying the nitrogen gas. Especially, even leakage of a small amount (about 0.1 sccm) can be detected quickly (within a few minutes) compared to a conventional leakage determination method for measuring a pressure in the vacuum transfer chamber TM (detection limit: about 0.9 sccm).

Under the condition in which there is no leakage (leakage amount: 0 sccm), the increase in the oxygen concentration was not monitored even after the stop of supplying the nitrogen gas. From the above, it is clear that whether or not the leakage occurs and, if occurs, whether or not the leakage amount exceeds an allowable level can be quickly determined by measuring the oxygen concentration after the supply of the nitrogen gas for pressure control is stopped.

(Test 2)

The oxygen concentration in the vacuum transfer chamber TM under the respective conditions were measured while varying a set pressure in the vacuum transfer chamber TM and a leakage amount.

A. Test Condition

As in the case of the test 1, the (simulated) leakage amount of atmospheric air was varied within a range from 1 sccm to 5 sccm, and a set pressure in the evacuated vacuum transfer chamber TM was varied to different levels of 26 Pa, 106 Pa and 260 Pa. Under the respective conditions, the oxygen concentration in the vacuum transfer chamber TM was read out when the change thereof became stable.

B. Test Result

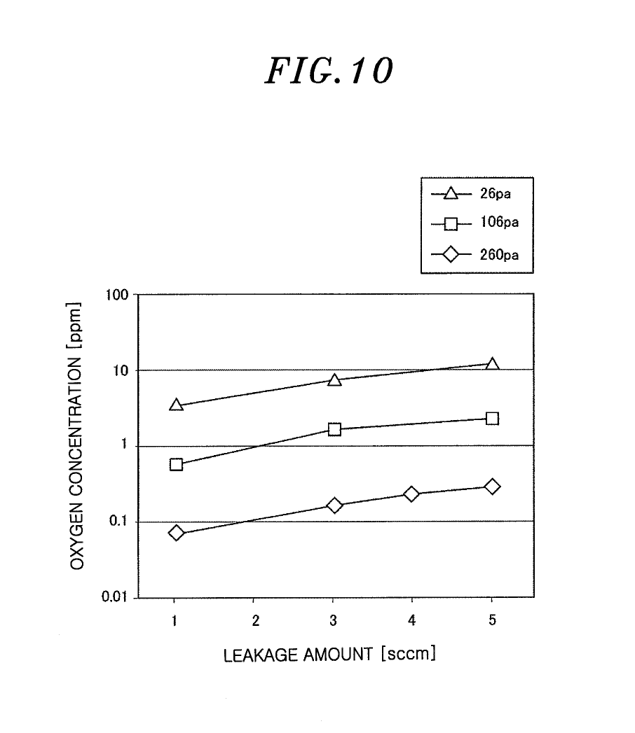

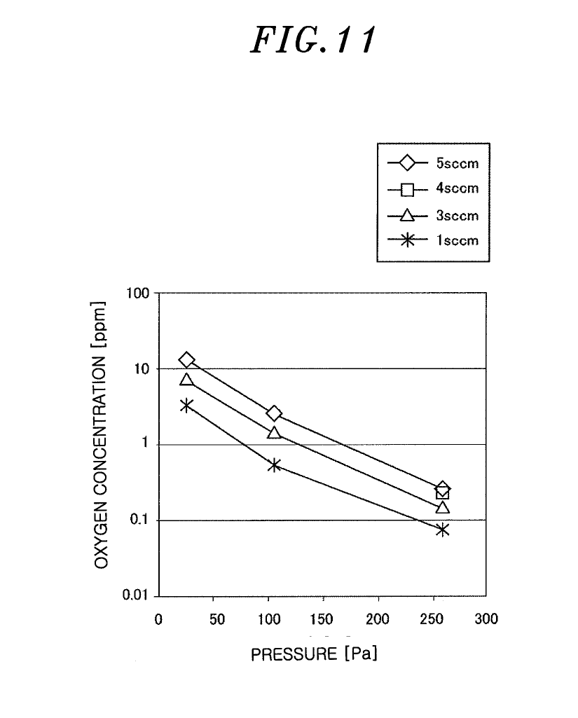

A test result is shown in FIGS. 10 and 11. In FIG. 10, the horizontal axis represents a leakage amount of atmospheric air and the vertical axis represents an oxygen concentration in the vacuum transfer chamber TM. Parameters 26 Pa, 106 Pa, and 260 Pa as the set pressure in the vacuum transfer chamber TM were plotted as different marks. In FIG. 11, the horizontal axis represents a set pressure in the vacuum transfer chamber TM and the vertical axis represents an oxygen concentration in the vacuum transfer chamber TM. Parameters 5 sccm, 4 sccm, 3 sccm, and 1 sccm as the leakage amount were plotted as different marks.

Referring to FIGS. 10 and 11, in the case of varying the set pressure in the vacuum transfer chamber TM and the leakage amount, the oxygen concentration in the vacuum transfer chamber TM is specified under the respective conditions. Since it is difficult to completely reduce the oxygen concentration in the vacuum transfer chamber TM to zero, a base oxygen concentration needs to be obtained when there is no leakage. The oxygen concentration is measured by the oxygen meter 24 during the operation of the substrate processing apparatus 1. When the measurement value exceeds a predetermined value, alarm is generated (referring to FIG. 11, for example, when the set pressure is 100 Pa, if the oxygen concentration in the vacuum transfer chamber TM is 1 ppm or above, it is possible to determine that the leakage amount exceeds 1 sccm). In that case, it is not required to stop the supply of the nitrogen gas or reduce the supply amount of the nitrogen gas.

While the disclosure has been shown and described with respect to the embodiments, it will be understood by those skilled in the art that various changes and modifications may be made without departing from the scope of the disclosure as defined in the following claims.

What is claimed is:

1. A leakage determining method for determining whether or not atmospheric air enters a vacuum transfer chamber for transferring a substrate under a vacuum atmosphere between at least one preliminary vacuum chamber and at least one processing chamber, the vacuum transfer chamber being connected to the preliminary vacuum chamber of which inner atmosphere is switchable between an atmospheric atmosphere and a vacuum atmosphere and to the processing chamber where the substrate is processed under a vacuum atmosphere, via respective opening/closing valves, the method comprising:
controlling, when the substrate is transferred, a pressure in the vacuum transfer chamber to a preset pressure by supplying a pressure control gas into the vacuum transfer chamber being evacuated;
performing supply control, when the substrate is not transferred, by reducing the amount of the pressure control gas supplied into the vacuum transfer chamber or stopping the supply of the pressure control gas; and
measuring with an oxygen meter an oxygen concentration in the vacuum transfer chamber after the supply control of the pressure control gas and determining leakage of atmospheric air into the vacuum transfer chamber by determining whether or not atmospheric air whose amount exceeds a preset allowable level enters the vacuum transfer chamber based on temporal changes of the measured oxygen concentration.

2. The method of claim 1, wherein the supply control of the pressure control gas is performed while evacuating the vacuum transfer chamber.

3. The method of claim wherein the oxygen concentration is measured in a state where the opening/closing valves provided between the preliminary vacuum chamber and the vacuum transfer chamber and between the processing chamber and the vacuum transfer chamber are closed.

4. The method of claim 1, wherein the oxygen concentration is measured in a state where the opening/closing valve provided between the vacuum transfer chamber and the preliminary vacuum chamber of a vacuum atmosphere is opened and the opening/closing valve provided between the vacuum transfer chamber and the processing chamber is closed.

5. The method of claim 4, wherein the at least one preliminary vacuum chamber includes a plurality of preliminary vacuum chambers, and the preliminary vacuum chambers are connected to the vacuum transfer chamber and the oxygen concentration is measured in a state where the opening/closing valve provided between the vacuum transfer chamber and one of the preliminary vacuum chambers is opened.

6. The method of claim 1, wherein the oxygen concentration is measured in a state where the opening/closing valve provided between the vacuum transfer chamber and the processing chamber is opened and the opening/closing valve provided between the vacuum transfer chamber and the preliminary vacuum chamber is closed.

7. The method of claim 6, wherein the at least one processing chamber includes a plurality of processing chambers, and the processing chambers are connected to the vacuum transfer chamber and the oxygen concentration is measured in a state where the opening/closing valve provided between the vacuum transfer chamber and one of the processing chambers is opened.

8. The method of claim 1, wherein a process performed in the processing chamber includes a substrate heating process.

9. The method of claim 1, wherein said performing supply control of the pressure control gas and said determining leakage of atmospheric air into the vacuum transfer chamber are performed during a period in which the substrate is not processed in the processing chamber.

10. The method of claim 1, wherein said performing supply control of the pressure control gas and said determining leakage of atmospheric air into the vacuum transfer chamber are performed when the substrate is not transferred between the preliminary vacuum chamber and the processing chamber during processing of the substrate in the processing chamber.

11. The method of claim 1, wherein the preset pressure in the vacuum transfer chamber is within a range from about 10 Pa to about 1333 Pa.

12. A substrate processing apparatus comprising:
a preliminary vacuum chamber of which inner atmosphere is switchable between an atmospheric atmosphere and a vacuum atmosphere;
a processing chamber where a substrate is processed under a vacuum atmosphere;
a vacuum transfer chamber connected to the preliminary vacuum chamber and the processing chamber via respective opening/closing valves, the vacuum transfer chamber including a transfer unit configured to transfer the substrate between the preliminary vacuum chamber and the processing chamber under a vacuum atmosphere obtained by evacuation;
a gas supply unit configured to supply a pressure control gas into the vacuum transfer chamber;
an oxygen meter configured to measure an oxygen concentration in the vacuum transfer chamber;
a control unit configured to output a control signal for controlling a pressure in the vacuum transfer chamber to a preset level by supplying a pressure control gas from the gas supply unit when the substrate is transferred, performing supply control by reducing the amount of the pressure control gas supplied to the vacuum transfer chamber or stopping the supply of the pressure control gas when the substrate is not transferred, and measuring an oxygen concentration in the vacuum transfer chamber by the oxygen meter and determining whether atmospheric air whose amount exceeds a preset allowable level enters the vacuum transfer chamber based on temporal changes of the measured oxygen concentration.

13. The apparatus of claim 12, wherein the supply control of the pressure control gas is performed while the vacuum transfer chamber is evacuated.

14. The apparatus of claim 12, wherein the oxygen concentration is measured in a state where the opening/closing valves provided between the preliminary vacuum chamber and the vacuum transfer chamber and between the processing chamber and the vacuum transfer chamber are closed.

15. The apparatus of claim 12, wherein processes performed in the processing chamber include a substrate heating process.

16. The apparatus of claim 12, wherein the preset pressure in the vacuum transfer chamber is within a range from about 10 Pa to about 1333 Pa.

17. A non-transitory storage medium storing a computer program which is used in a substrate processing apparatus for processing a substrate,
wherein the program has the steps of performing the leakage determination method described in claim 1.
